(19) United States
(12) Patent Application Publication
(10) Pub. No.: US 2021/0264808 A1
(43) Pub. Date: Aug. 26, 2021

(54) AD-HOC TRAINING INJECTION BASED ON USER ACTIVITY AND UPSKILLING SEGMENTATION

(71) Applicant: INTERNATIONAL BUSINESS MACHINES CORPORATION, ARMONK, NY (US)

(72) Inventors: Mary Rudden, Denver, CO (US); Craig M. Trim, Ventura, CA (US); Zachary A. Silverstein, Jacksonville, FL (US); Jeremy R. Fox, Georgetown, TX (US)

(21) Appl. No.: 16/795,997

(22) Filed: Feb. 20, 2020

(51) Int. Cl. G09B 19/00; G16H 50/20; G06Q 10/10; G16H 40/67; G06N 20/00; A61B 5/16

(52) U.S. Cl. CPC G09B 19/00; G16H 50/20; G06Q 10/1097; A61B 5/167; G06N 20/00; A61B 5/165; G16H 40/67

(57) ABSTRACT

A method, computer system, and computer program product for ad-hoc training injection are provided. The embodiment may include receiving a user learning plan. The embodiment may also include processing the received user learning plan by dividing content into smaller pieces. The embodiment may further include determining a current user mental state. The embodiment may also include determining an optimum time for a user to engage in the learning plan. The embodiment may further include presenting one of the smaller pieces at the determined optimum time.

### BACKGROUND

The present invention relates, generally, to the field of computing, and more particularly to a training injection based on user activity and upskilling segmentation utilizing AI-based learning management systems.

Learning management systems relate to an application for the administration, documentation, tracking, reporting, and delivery of educational courses, training programs, or learning and development programs. Learning management systems were designed to identify training and learning gaps, utilizing analytical data and reporting. Learning management systems are used to deploy a variety of learning strategies across different formats, including formal, experiential and social learning to manage functions such as compliance training, certification management, and sales enablement. An AI engine may help personalize the learning experience for each learner by offering course formats based on their learning interests or capabilities. Such AI engine-based learning management systems may also suggest additional or follow-up courses with topics most relevant to the learners' past learning activities.

### SUMMARY

According to one embodiment, a method, computer system, and computer program product ad-hoc training injection are provided. The embodiment may include receiving a user learning plan. The embodiment may also include processing the received user learning plan by dividing content into smaller pieces. The embodiment may further include determining a current user mental state. The embodiment may also include determining an optimum time for a user to engage in the learning plan. The embodiment may further include presenting one of the smaller pieces at the determined optimum time.

### BRIEF DESCRIPTION OF THE SEVERAL VIEWS OF THE DRAWINGS

These and other objects, features, and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating an ad-hoc training injection process according to at least one embodiment;

FIG. 3 is a block diagram showing an exemplary process of dividing learning content into digestible pieces utilizing an ad-hoc training injection process according to at least one embodiment;

FIG. 4 is a block diagram showing an exemplary determination process of a user's best historical productivity on learning materials according to at least one embodiment;

FIG. 5 is a block diagram of internal and external components of computers and servers depicted in FIG. 1 according to at least one embodiment;

FIG. 6 depicts a cloud computing environment according to an embodiment of the present invention; and

FIG. 7 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to a training injection based on user activity and upskilling segmentation utilizing AI-based learning management systems. The following described exemplary embodiments provide a system, method, and program product to capture a given user's learning plans and divide the learning materials into easily digestible chunks. The following described exemplary embodiment also provides a system, method, and program product to monitor the user's current ongoing activity and inject fragments into regular business throughout the day. Therefore, the present embodiment has the capacity to improve the technical field of learning management systems by using a computer processor to divide current learning content into small groups of learning content based on analysis of various user data and determine a optimum time for injection of the divided learning content such that the user may digest the given pieces of learning content more efficiently.

As previously described, learning management systems relate to an application for the administration, documentation, tracking, reporting, and delivery of educational courses, training programs, or learning and development programs. Learning management systems were designed to identify training and learning gaps, utilizing analytical data and reporting. Learning management systems are used to deploy a variety of learning strategies across different formats, including formal, experiential and social learning to manage functions such as compliance training, certification management and sales enablement. An AI engine may help personalize the learning experience for each learner by offering course formats based on their learning interests or capabilities. Such AI engine-based learning management systems may also suggest additional or follow-up courses with topics most relevant to the learners' past learning activities.

Learning management systems were designed to identify training and learning gaps, utilizing analytical data and reporting. Learning management systems are focused on online learning delivery but support a range of uses, acting as a platform for online content, including courses, both asynchronous-based and synchronous-based. In interacting with digital devices, there is almost always something new to learn. Large enterprises often have recommended coursework. Similarly, an individual may be interested in learning any given topic. Effective engagement requires high activity levels and concerted effort from the user. It may involve stopping a current activity, scheduling time, or clearing up a schedule. An educational goal tends to become a non-primary goal in such cases. As such, it may be advantageous to, among other things, implement a system capable of providing a solution to help inject ad-hoc training activities of learning content in easily digestible parts.

According to one embodiment, the present invention may define learning-based educational requirements and conduct iterative, incremental upskilling delivery to users based on an AI model. In at least one other embodiment, the present invention may deliver a model to produce AI-based upskilling content segmentation to provide progressive education to a user. According to one other embodiment, the present invention may utilize AI to adjust the size, scope, and delivery mechanism for the upskilling education segmented content to the user.

The present invention may be a system, a method, and/or a computer program product at any possible technical detail level of integration. The computer program product may include the computer readable storage medium (or media) having computer readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer-readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or another device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product for analyzing a user's learning activities and learning content to create personalized content pieces that are more easily digestible by the user.

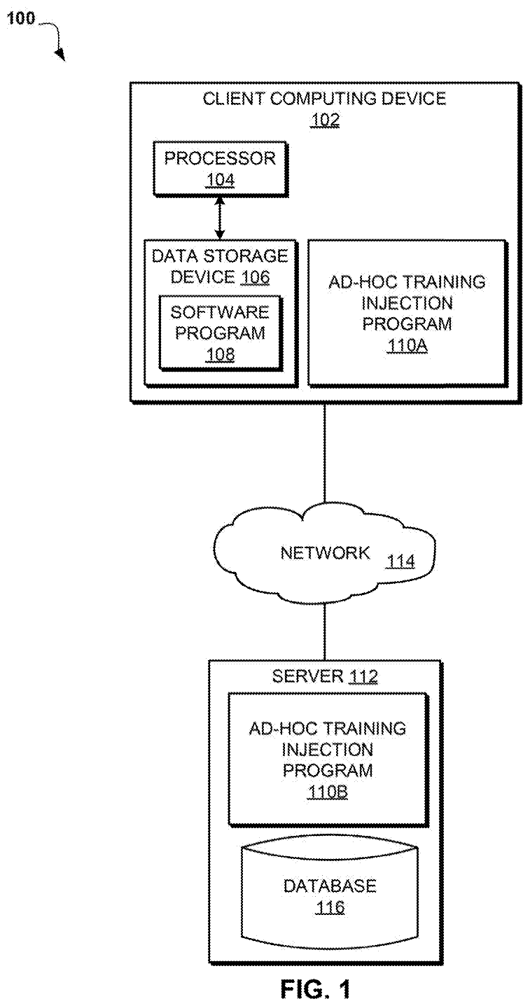

Referring to FIG. 1, an exemplary networked computer environment 100 is depicted, according to at least one embodiment. The networked computer environment 100 may include client computing device 102 and a server 112 interconnected via a communication network 114. According to at least one implementation, the networked computer environment 100 may include a plurality of client computing devices 102 and servers 112 of which only one of each is shown for illustrative brevity.

The communication network 114 may include various types of communication networks, such as a wide area network (WAN), local area network (LAN), a telecommunication network, a wireless network, a public switched network and/or a satellite network. The communication network 114 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

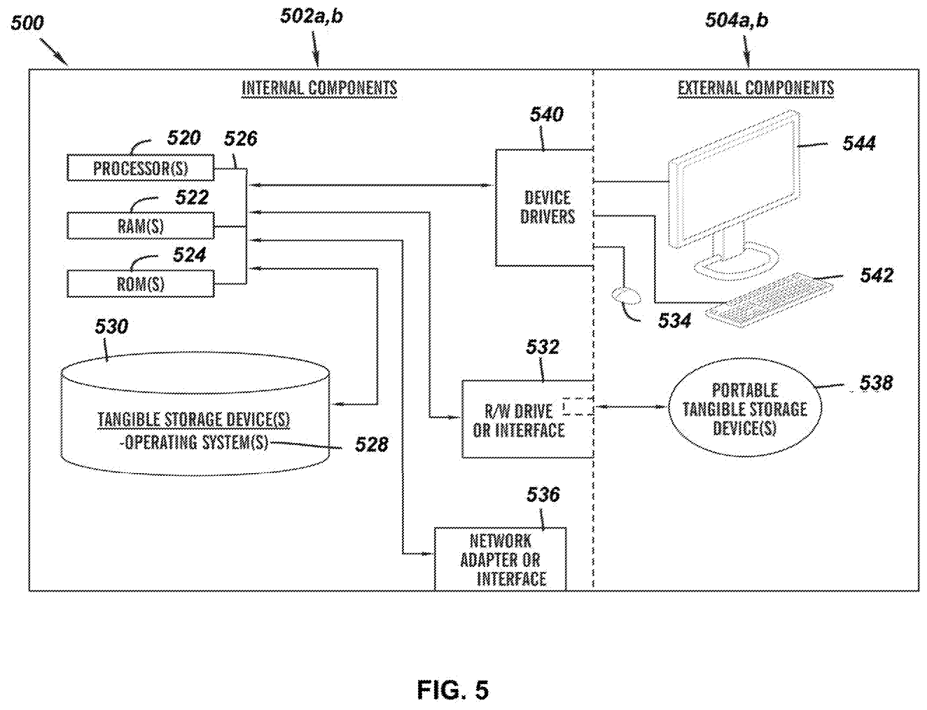

Client computing device 102 may include a processor 104 and a data storage device 106 that is enabled to host and run a software program 108 and an ad-hoc training injection program 110A and communicate with the server 112 via the communication network 114, in accordance with one embodiment of the invention. Client computing device 102 may be, for example, a mobile device, a telephone, a personal digital assistant, a netbook, a laptop computer, a tablet computer, a desktop computer, or any type of computing device capable of running a program and accessing a network. As will be discussed with reference to FIG. 5, the client computing device 102 may include internal components 502a and external components 504a, respectively.

The server computer 112 may be a laptop computer, netbook computer, personal computer (PC), a desktop computer, or any programmable electronic device or any network of programmable electronic devices capable of hosting and running an ad-hoc training injection program 110B and a database 116 and communicating with the client computing device 102 via the communication network 114, in accordance with embodiments of the invention. As will be discussed with reference to FIG. 5, the server computer 112 may include internal components 502b and external components 504b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

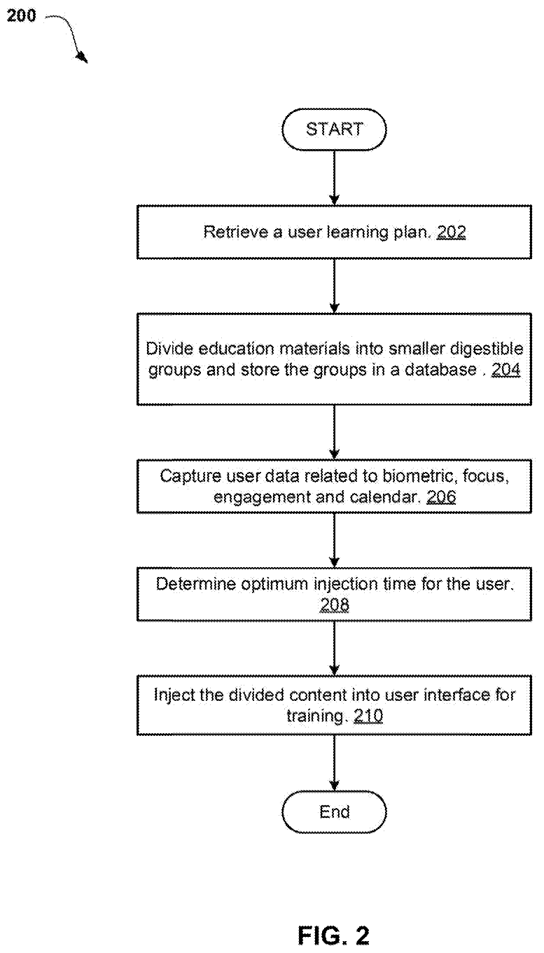

According to the present embodiment, the ad-hoc training injection program 110A 110B may be a program capable of capturing user's current mental state, device activity, schedule and learning plan using computer programs or applications embedded in the client computing device 102. The social analytics petition creation program 110A, 110B may also process the user's learning plan and grouping the user's learning content into digestible pieces based on complexity, size, and activities. The ad-hoc training injection process is explained in further detail below with respect to FIG. 2.

FIG. 2 is an operational flowchart illustrating an ad-hoc training injection process 200 according to at least one embodiment. At 202, the ad-hoc training injection program 110A, 110B retrieves a user's learning plan. According to one embodiment, the ad-hoc training injection program 110A, 110B may prompt a user to opt into a training injection module that may capture a user's current mental state, schedule, activity and learning plan. The ad-hoc training injection program 110A, 110B may interact with the client computing device 102 to retrieve a user's current schedule and job-related activities based on the user's email, social media applications and calendar applications. The ad-hoc training injection program 110A, 110B may also retrieve information related to the user's mental state based on AI-based analysis of the user's recent schedules (e.g. daily tasks, projects, business trips, sick days, vacation days, etc.) retrieved from the user's emails, calendar applications and social media applications. Learning plans, for example, may relate to a user workplace's employees upskilling plans or requirements announced in advance through the workplace's portal or email announcement sent out to the employees. The user's learning plan may also relate to the user's personal learning plan outside the user's job function or responsibility. Such plans may be retrieved from the user's emails or calendar as well. Next, the ad-hoc training injection program 110A, 110B may retrieve appropriate learning materials from education courses in which the user may have enrolled. In one embodiment, such learning materials may be any type of learning material in any format, such as video, pictures, text or audio format.

At 204, the ad-hoc training injection program 110A, 110B divides education materials into smaller digestible groups and store the groups in a database. According to one embodiment, the ad-hoc training injection program 110A, 110B may process the user's learning plans and divide the retrieved learning content into digestible chunks or smaller groups based on size, complexity, and length. Learning content may be analyzed using any known image recognition, sound recognition and natural language processing techniques. In one embodiment, chunks may be a set size based on module or user preference. For example, a user may manually configure the size or length of each chunk to be less than 5 minutes. Size may be determined based on word count or average expected reading length. Complexity may be determined based on a score depending on the complexity of the words used in the learning content or technical difficulty of the concept or complexity of technical words. The ad-hoc training injection program 110A, 110B may determine activities required within each learning content, such as quiz, homework, project or specific activities like coding activity. Depending on difficulties or expected length for each activity, the ad-hoc training injection program 110A, 110B may adjust the size of each chunk or small group of learning content.

In at least one other embodiment, the chunking process mentioned above may be implemented using an exemplary algorithm described below.

The ad-hoc training injection program 110A, 110B may begin with some initial value X⁽ⁱ⁾ and the next sample may be X⁽ⁱ⁺¹⁾. Since X⁽ⁱ⁺¹⁾=(x₁⁽ⁱ⁺¹⁾, x₂⁽ⁱ⁺¹⁾, . . . , xₙ⁽ⁱ⁺¹⁾ is a vector, the system sample each component of the vector xⱼ⁽ⁱ⁺¹⁾ from the distribution of that component conditioned on all other components sampled so far. The ad-hoc training injection program 110A, 110B may specify the pseudo-code interpretation of this equation as X\hat{ }((i+1))=(x_1\hat{ }((i+1)),x_2\hat{ }((i+1)), . . . , x_n\hat{ }((i+1)). The ad-hoc training injection program 110A, 110B may condition on the X⁽ⁱ⁺¹⁾ component up to xⱼ₋₁⁽ⁱ⁺¹⁾. The ad-hoc training injection program 110A, 110B may then condition on the X⁽ⁱ⁾ component, starting from xⱼ₊₁⁽ⁱ⁾ to xₙ⁽ⁱ⁾.

In order to achieve the sequence described, the ad-hoc training injection program 110A, 110B may sample the components in order, starting from the first component. This may imply that when the system samples xⱼ⁽ⁱ⁺¹⁾ it will update the values based on the distribution specified by p(xⱼ⁽ⁱ⁺¹⁾, . . . , xⱼ₋₁⁽ⁱ⁺¹⁾, xⱼ₊₁⁽ⁱ⁾, . . . , xₙ⁽ⁱ⁾. The ad-hoc training injection program 110A, 110B may specify the pseudo-code interpretation of this equation as p(x_j\hat{ }((i+1))┤|x_1\hat{ }((i+1)), . . . , x_(j−1)\hat{ }((i+1)), x_(j+1)\hat{ }((i)), . . . , x_n\hat{ }((i)) for easier implementation. The above steps may be repeated according to parameter k times.

At 206, the ad-hoc training injection program 110A, 110B captures a user's biometric, focus, engagement, and calendar. According to one embodiment, the ad-hoc training injection program 110A, 110B may interact with the client computing device 102 to retrieve a user's current schedule and job-related activities based on the user's email, social media applications and calendar applications. The ad-hoc training injection program 110A, 110B may also retrieve information related to the user's mental state based on AI-based analysis of the user's recent schedules (e.g. daily tasks, projects, business trips, sick days, vacation days, etc.) retrieved from the user's emails, calendar applications and social media applications. In yet another embodiment, the ad-hoc training injection program 110A, 110B may capture a user's biometric information from a wearable device that the user frequently uses, such as a smartwatch or other types of biometric measuring devices that the user daily uses, such as a portable blood pressure reader, etc. For example, the ad-hoc training injection program 110A, 110B may determine that a user is under stress based on the tight job task schedules indicated on the calendar or biometric information indicating that the user may have been suffering from relatively high blood pressure due to recent stress at work. All these factors may be taken into account to determine the optimal timing of learning content injection in the next step.

At 208, the ad-hoc training injection program 110A, 110B determines an optimum injection time for the user. According to one embodiment, the ad-hoc training injection program 110A, 110B may capture an optimum time to inject an activity based on the user's activity pattern or based on a predefined threshold set by a user. For example, a user may predefine the threshold to be free time without any schedule during the day for at least one hour. In another example, the ad-hoc training injection program 110A, 110B may determine an optimum time based on the user's previous activities and results that the user obtained from such activities in the past (e.g. activity time, score on the quiz, or final pass or fail). The user's activity pattern may also be taken into consideration when determining the optimum time. For example, if the user's activity pattern indicates, the user tends to start and complete any required learning activity at work at the end of each quarter, the ad-hoc training injection program 110A, 110B may wait until the end of the quarter to inject any learning materials.

At 210, the ad-hoc training injection program 110A, 110B injects the divided content into a user interface for training. According to one embodiment, the ad-hoc training injection program 110A, 110B may inject learning content as a push notification, user interface artifact pop-up or any other type of interactable event on the user computing device 102. In at least one other embodiment, the ad-hoc training injection program 110A, 110B may inject one small group or chunk of learning content and gauge the user's interaction with the injected learning content for learning loop and self-optimization, such that the ad-hoc training injection program 110A, 110B may adjust the next injection time. For example, if a user receives the first part of the divided learning content and delays completing the learning material, the ad-hoc training injection program 110A, 110B may try a different injection time to check whether the user delays the learning process repeatedly

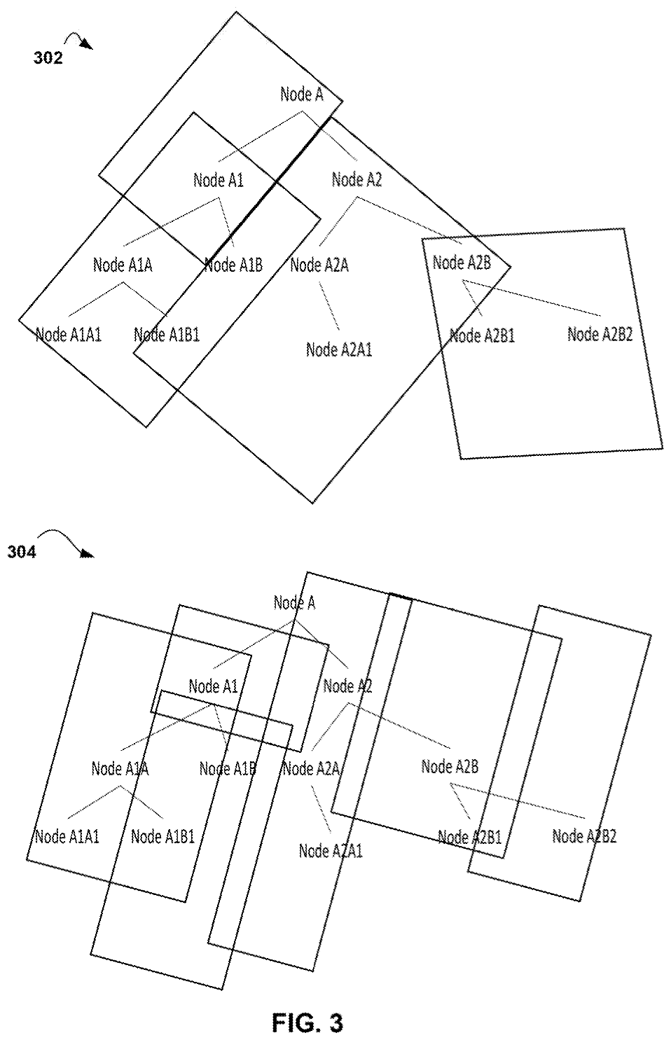

Referring now to FIG. 3, a block diagram showing an exemplary process of dividing learning content into digestible pieces utilizing an ad-hoc training injection process is depicted according to at least one embodiment. According to one embodiment, the ad-hoc training injection program 110A, 110B may analyze keywords, explicit metadata (e.g. table of contents, index, hyperlinks, etc.), and implicit metadata (e.g. length of content, user activity, number of times a module is watched, etc.) to obtain latent taxonomy of learning content. In at least one embodiment, the ad-hoc training injection program 110A, 110B may perform a splitting using an algorithm, rather than presenting learning content all at once to a user. In FIG. 3, two conceptual chunking or dividing methods are depicted according to one embodiment. Diagram 302 demonstrates the content being chunked for a user who has some background familiarity with topics contained in the content. For example, the size of the middle chunk in Diagram 302 may indicate the relative ease with which the ad-hoc training injection program 110A, 110B determines the user may be able to digest this chunk, based on background and prior skills. Diagram 304 may represent a bifurcation strategy for a user with little to no background in the space. The chunking in Diagram 304 may have far greater overlaps, and the middle portion may be broken down further for a user with less background familiarity.

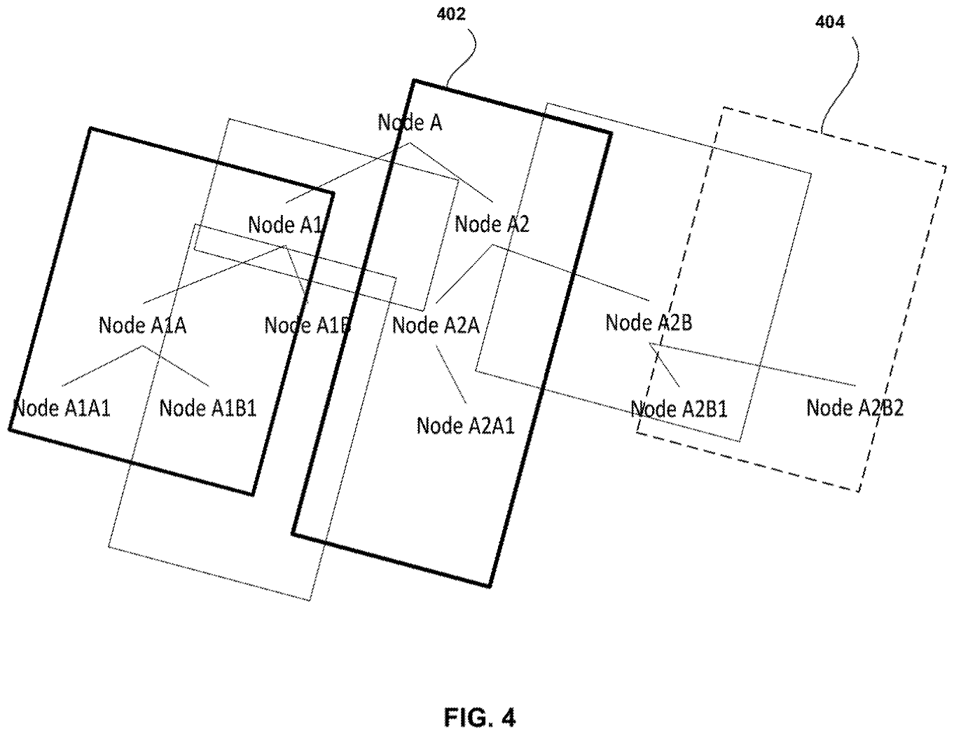

Referring now to FIG. 4, a block diagram showing an exemplary determination process of a user's best historical productivity on learning materials is depicted according to at least one embodiment. According to one embodiment, the ad-hoc training injection program 110A, 110B may determine the user's best historical productivity on learning materials based on the machine-learned optimization process. In this diagram, the solid lines surrounding the box 402 may represent content that has a high historical pattern of low productivity, and thus, the ad-hoc training injection program 110A, 110B may need to either present these chunks multiple times or allocate more time for them. On the other hand, a chink with a dotted line or lower line weight surrounding the box 404 may present these chunks with less time in between as the user has more familiarity. The user may have an easier time digesting the content and may move on to the next content more quickly. In at least one other embodiment, the ad-hoc training injection program 110A, 110B may allow a user to adjust the chunks to take into account the user's personal learning pattern.

It may be appreciated that FIGS. 2-4 provide only an illustration of one implementation and do not imply any limitations with regard to how different embodiments may be implemented. Many modifications to the depicted environments may be made based on design and implementation requirements. For example, in at least one embodiment, the ad-hoc training injection program 110A, 110B may crowdsource approval or disapproval for size and brevity of delivery in regard to divided groups of learning content. The ad-hoc training injection program 110A, 110B may allow a user to rate the user's ability to consume the AI-based divided content and also crowdsource other user's ratings with respect to the similar learning content to improve the system's chunking process.

FIG. 5 is a block diagram 500 of internal and external components of the client computing device 102 and the server 112 depicted in FIG. 1 in accordance with an embodiment of the present invention. It should be appreciated that FIG. 5 provides only an illustration of one implementation and does not imply any limitations with regard to the environments in which different embodiments may be implemented. Many modifications to the depicted environments may be made based on design and implementation requirements.

The data processing system 502, 504 is representative of any electronic device capable of executing machine-readable program instructions. The data processing system 502, 504 may be representative of a smartphone, a computer system, PDA, or other electronic devices. Examples of computing systems, environments, and/or configurations that may represented by the data processing system 502, 504 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 502 a,b and external components 504 a,b illustrated in FIG. 5. Each of the sets of internal components 502 include one or more processors 520, one or more computer-readable RAMs 522, and one or more computer-readable ROMs 524 on one or more buses 526, and one or more operating systems 528 and one or more computer-readable tangible storage devices 530. The one or more operating systems 528, the software program 508 and the ad-hoc training injection program 110A in the client computing device 102 and the ad-hoc training injection program 110B in the server 112 are stored on one or more of the respective computer-readable tangible storage devices 530 for execution by one or more of the respective processors 520 via one or more of the respective RAMs 522 (which typically include cache memory). In the embodiment illustrated in FIG. 5, each of the computer-readable tangible storage devices 530 is a magnetic disk storage device of an internal hard drive. Alternatively, each of the computer-readable tangible storage devices 530 is a semiconductor storage device such as ROM 524, EPROM, flash memory or any other computer-readable tangible storage device that can store a computer program and digital information.

Each set of internal components 502 a,b also includes an R/W drive or interface 532 to read from and write to one or more portable computer-readable tangible storage devices 538 such as a CD-ROM, DVD, memory stick, magnetic tape, magnetic disk, optical disk or semiconductor storage device. A software program, such as the ad-hoc training injection program 110A,110B can be stored on one or more of the respective portable computer-readable tangible storage devices 538, read via the respective R/W drive or interface 532 and loaded into the respective hard drive 530.

Each set of internal components 502 a,b also includes network adapters or interfaces 536 such as a TCP/IP adapter cards, wireless Wi-Fi interface cards, or 3G or 4G wireless interface cards or other wired or wireless communication links. The software program 108 and the ad-hoc training injection program 110A in the client computing device 102 and the ad-hoc training injection program 110B in the server 112 can be downloaded to the client computing device 102 and the server 112 from an external computer via a network (for example, the Internet, a local area network or other, wide area network) and respective network adapters or interfaces 536. From the network adapters or interfaces 536, the software program 108 and the ad-hoc training injection program 110A in the client computing device 102 and the ad-hoc training injection program 110B in the server 112 are loaded into the respective hard drive 530. The network may comprise copper wires, optical fibers, wireless transmission, routers, firewalls, switches, gateway computers and/or edge servers.

Each of the sets of external components 504 a,b can include a computer display monitor 544, a keyboard 542, and a computer mouse 534. External components 504 a,b can also include touch screens, virtual keyboards, touch pads, pointing devices, and other human interface devices. Each of the sets of internal components 502 a,b also includes device drivers 540 to interface to computer display monitor 544, keyboard 542, and computer mouse 534. The device drivers 540, R/W drive or interface 532, and network adapter or interface 536 comprise hardware and software (stored in storage device 530 and/or ROM 524).

It is understood in advance that although this disclosure includes a detailed description on cloud computing, implementation of the teachings recited herein is not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, andcompliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is a service oriented with a focus on statelessness, low coupling, modularity, and semantic interoperability. At the heart of cloud computing is an infrastructure comprising a network of interconnected nodes.

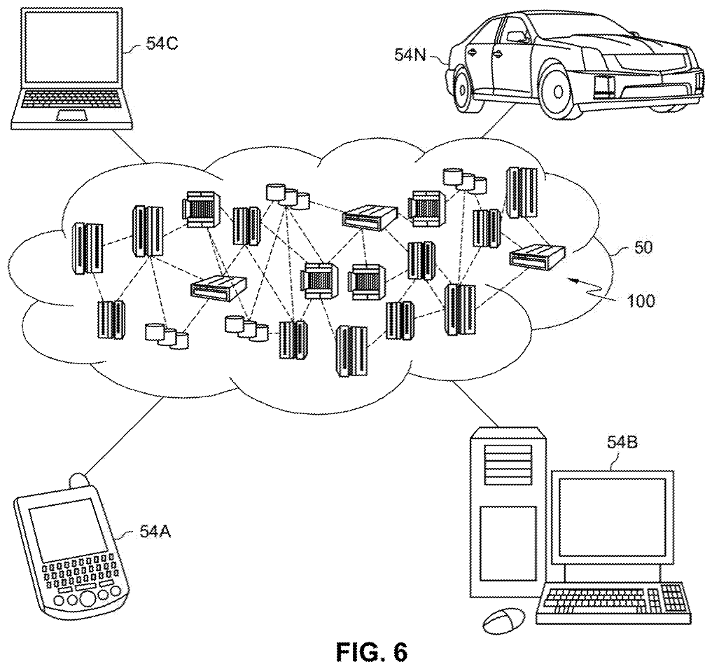

Referring now to FIG. 6, illustrative cloud computing environment 50 is depicted. As shown, cloud computing environment 50 comprises one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 100 and cloud computing environment 50 can communicate with any type of computerized device over any type of network and/or network addressable connection (e.g., using a web browser).

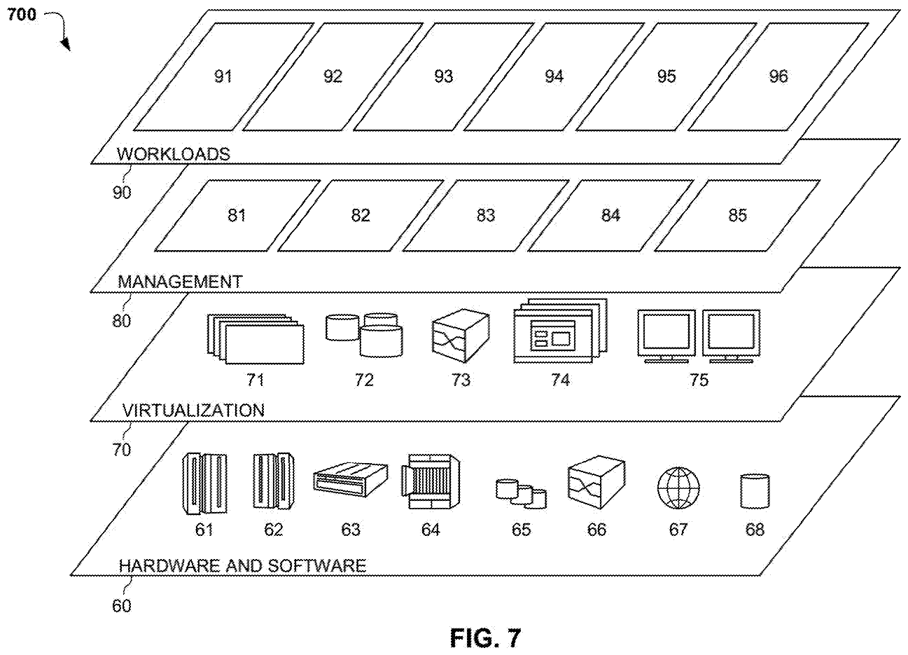

Referring now to FIG. 7, a set of functional abstraction layers 700 provided by cloud computing environment 50 is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and ad-hoc training injection 96. Ad-hoc training injection 96 may relate to receiving a user's learning plan and processing the received user's learning plan by dividing content into digestible pieces.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for ad-hoc training injection, the method comprising:
receiving a user learning plan;
processing the received user learning plan by dividing learning content into smaller pieces;
determining a current user mental state;
determining an optimum time for a user to engage in the user learning plan; and
presenting one of the smaller pieces at the determined optimum time.

2. The method of claim 1, wherein dividing the content into digestible pieces is based on size or complexity of the learning content or user daily activities using a chunking algorithm.

3. The method of claim 1, wherein the current user state is determined based on captured user biometric data.

4. The method of claim 1, wherein the current user state is determined based on analysis of a user schedule retrieved from user calendar, emails or social media applications.

5. The method of claim 1, further comprising:
receiving feedback from the user with respect to the determined optimum time; and
adjusting a time to present another digestible piece.

6. The method of claim 1, further comprising:
receiving feedback from the user with respect to the digestible pieces; and
adjusting a size or a length of another digestible piece to be presented to the user.

7. The method of claim 1, further comprising:
determining a user best historical productivity value for similar previous learning materials.

8. A computer system for ad-hoc training injection, the computer system comprising:
one or more processors, one or more computer-readable memories, one or more computer-readable tangible storage media, and program instructions stored on at least one of the one or more tangible storage media for execution by at least one of the one or more processors via at least one of the one or more memories, wherein the computer system is capable of performing a method comprising:
receiving a user learning plan;
processing the received user learning plan by dividing learning content into smaller pieces;
determining a current user mental state;
determining an optimum time for a user to engage in the user learning plan; and
presenting one of the smaller pieces at the determined optimum time.

9. The computer system of claim 8, wherein dividing the content into digestible pieces is based on size or complexity of the learning content or user daily activities using a chunking algorithm.

10. The computer system of claim 8, wherein the current user state is determined based on captured user biometric data.

11. The computer system of claim 8, wherein the current user state is determined based on analysis of a user schedule retrieved from user calendar, emails or social media applications.

12. The computer system of claim 8, further comprising:
receiving feedback from the user with respect to the determined optimum time; and
adjusting a time to present another digestible piece.

13. The computer system of claim 8, further comprising:
receiving feedback from the user with respect to the digestible pieces; and
adjusting a size or a length of another digestible piece to be presented to the user.

14. The computer system of claim 8, further comprising:
determining a user best historical productivity value for similar previous learning materials.

15. A computer program product for ad-hoc training injection, the computer program product comprising:
one or more computer-readable tangible storage media and program instructions stored on at least one of the one or more tangible storage media, the program instructions executable by a processor of a computer to perform a method, the method comprising:
receiving a user learning plan;
processing the received user learning plan by dividing learning content into smaller pieces;
determining a current user mental state;
determining an optimum time for a user to engage in the user learning plan; and
presenting one of the smaller pieces at the determined optimum time.

16. The computer program product of claim 15, wherein dividing the content into digestible pieces is based on size or complexity of the learning content or user daily activities using a chunking algorithm.

17. The computer program product of claim 15, wherein the current user state is determined based on captured user biometric data.

18. The computer program product of claim 15, wherein the current user state is determined based on analysis of a user schedule retrieved from user calendar, emails or social media applications.

19. The computer program product of claim 15, further comprising:
receiving feedback from the user with respect to the determined optimum time; and
adjusting a time to present another digestible piece.

20. The computer program product of claim 15, further comprising:
receiving feedback from the user with respect to the digestible pieces; and
adjusting a size or a length of another digestible piece to be presented to the user.
